(19) United States
(12) Patent Application Publication
(10) Pub. No.: US 2014/0271454 A1
(43) Pub. Date: Sep. 18, 2014

(54) CELL-SYNTHESIZED PARTICLES

(71) Applicants: Nicolas L'Heureux, San Rafael, CA (US); Todd N McAllister, San Anselmo, CA (US)

(72) Inventors: Nicolas L'Heureux, San Rafael, CA (US); Todd N McAllister, San Anselmo, CA (US)

(73) Assignee: CYTOGRAFT TISSUE ENGINEERING, INC., Novato, CA (US)

(21) Appl. No.: 14/113,060

(22) PCT Filed: Apr. 23, 2012
(86) PCT No.: PCT/US12/34692

§ 371 Date: May 31, 2014

Related U.S. Application Data
(60) Provisional application No. 61/478,033, filed on Apr. 21, 2011.

(51) Int. Cl. A61L 27/36; A61K 45/06

(52) U.S. Cl. CPC A61L 27/3633; A61L 27/3691; A61K 45/06
USPC 424/1.11; 424/93.7; 424/520; 424/93.21; 424/130.1; 424/489

(57) ABSTRACT

The present technology provides for a cell-synthesized biological particle, the processes for making a cell-synthesized particle, and a process for assembling such cell-synthesized particles into tissues and organs. The particles are synthesized in culture in vitro by living cells that produce natural extracellular matrix. Multiple particles can be assembled into tissues that have significant void space. The particles or tissues can act as a substrate for additional cell types. The particles or tissues can be further cultured in vitro to achieve favorable cell or extracellular matrix growth, organization or other desired characteristics. The particles or tissues can be devitalized or decellularized. The particles or tissues can be injected or implanted in a human to repair, enhance, or create a secretory, mechanical, or aesthetic function.

### CLAIM OF PRIORITY

This application claims the benefit of priority under 35 U.S.C. §119(e) to U.S. provisional application Ser. No. 61/478,033, filed Apr. 21, 2011, the content of which is incorporated herein by reference in its entirety.

### TECHNICAL FIELD

The present technology relates to the field of tissue engineering. More specifically, the technology relates to the field of particles comprising natural extracellular matrix produced from cells in culture, their methods of preparation and their subsequent uses.

### BACKGROUND

Multicellular organisms are made up of tissues, that is, groups of cells of similar form and function. Different tissues are assembled with one another and function together to form organs. While some tissues appear to be made exclusively from cells, most tissues have a second critical component—the extracellular matrix (ECM). The ECM is composed of various proteins that are synthesized by the cells and create the physical surrounding of the cell. The ECM plays a critical role in providing mechanical structure and also in providing adhesion sites for appropriate cell behavior, storage of growth factors and cytokines as well as other roles that are the subject of on-going research. Among the many proteins of the ECM, collagen is typically the most abundant and is well known for its mechanical role. About twenty types of collagens have been described to this day. Elastin is also a well-known protein that plays an important mechanical role in some tissues. The ECM also contains many other proteins that have various functions and create a complex micro-environment that cannot easily be reproduced artificially. (Those proteins include: fibronectins, laminins, vitronectin, decorin, fibulins, fibrillins, microfibril-associated glycoproteins (MFAP), emilins, osteopontin, tenascin, entactin/nidogen, thrombospondin, secreted proteins acidic and rich in cysteine (SPARC), matrilins, versican, hyaluronan, dermatan sulfate, heparan sulfate, chondroitin sulfate, keratan sulfate, biglycan, perlecan, syndecan, lumican, glypican, and many others). It is now well accepted that a properly-constituted ECM is critical to maintaining normal cell phenotype and function.

Tissue defects can arise from a number of medical conditions however, including, for example, congenital malformations, traumatic injuries, infections, and oncologic resections. For hundreds of years, surgeons have addressed the problem of repairing damaged tissue in living organisms. Transplantation of tissues and organs can provide solutions to many pathological conditions. However, the limited number of organ donors, and the limited ability of the body to regenerate, has left many patients untreated, often leading to much suffering and even death.

Successful repair of defective or damaged tissue depends in part on providing conditions that allow for appropriate cellular regeneration and that minimize the likelihood of infection and inflammation during the repair process. Because many tissues and organs cannot regenerate spontaneously, or cannot be repaired using existing surgical and pharmacological technologies, there is a continuing need for therapeutic strategies that promote tissue regeneration or that provide new tissue produced in vitro.

Tissue engineering emerged from a convergence of the fields of cell biology and engineering. Tissue engineering includes techniques and methods for producing tissues outside the human body, such as in vitro. Traditionally, tissue engineering has attempted to solve the problems involved in tissue repair using the principles of cell biology in conjunction with the techniques of material science to produce complex medical devices. Typically, tissue-engineering refers to construction of pieces of tissue from cellular components by self-assembly.

Nevertheless, artificial materials are often recognized by the body as a foreign object and cause an immune response, known as “foreign body reaction”, characterized by chronic inflammation and scarring (see, for example, Anderson, J. M., et al., Semin. Immunol. 20 (2), 86 (2008)). This response often results in the partial or total encapsulation of the artificial implant into scar tissue, which effectively isolates the implant from the circulatory system of the body. While this does not preclude many mechanical functions of the implant (as with, for example, orthopedic and dental implants), it does negatively affect the survival of cells located within the implant (a problem for, e.g., liver, kidney, pancreas replacements). This type of difficulty is particularly problematic for implants that are thick and contain large numbers of cells that require effective blood supply to bring oxygen and drain metabolic waste products. Furthermore, synthetic materials are prone to infections because they can harbor pathogenic microorganisms in a micro-environment that is difficult for the immune cells of the body to access. For these reasons, more recent approaches have focused on the expectation that the use of completely biological materials will allow for greater success in developing therapeutic strategies aimed at the repair, replacement, maintenance and enhancement of tissue function. However, development of purely biological materials is not without its own obstacles.

One approach to make fully biological tissue-engineered constructs has been to use proteins (such as collagen, elastin, and fibrin) that are extracted from animal or human sources. One can also use recombinant proteins obtained by biotechnological approaches. By physicochemical methods, these proteins can be assembled into different shapes (films, sponges, fibers, tubes, etc.). Particular cells can then be added to the proteinaceous structures to mimic the physiological organization or function of a specific tissue. One of the key limitations of this approach is the fact that proteins are often denatured or chemically modified by the extraction process needed to obtain the more or less purified proteins prior to their assembly into engineered constructs. When reconstituted into a tissue in vitro, these modified proteins lack the microstructure of the original proteins, or lack one or more required associated proteins, needed to recreate the micro-organization of the extracellular matrix (ECM). In most cases, even an intact protein will fail to reassemble spontaneously into the complex organization observed in nature, without the intervention of living cells. The abnormal microstructure of extracted proteins is then recognized by the non-specific immune system of the host as a “damaged” tissue. This leads to rapid degradation, loss of function and mechanical strength of this type of implant (see, e.g., Patino, M. G., et al., J. Oral Implantol. 28 (5), 220, (2002)). If the proteins are of animal origin and used in humans, they can also trigger recognition by the specific immune system and lead to rejection that way. Chemical treatments can be used to increase the mechanical strength of the construct or to render the proteins more resistant to enzymatic degradation, but this type of treatment further denatures the proteins and can trigger the foreign body reaction inflammatory process that the use of natural proteins aimed at avoiding in the first place (see, e.g., Cheung, D. T., et al., Connect. Tissue Res., 25 (1), 27 (1990)).

It has been observed that during long-term culture in the presence of ascorbic compounds, adherent cells, such as fibroblasts, can lay down large amounts of ECM proteins on the internal surfaces of culture dishes. As a result, living tissue, comprised of the living cells and the ECM proteins produced by the cells, can be detached from the culture dishes in planar shaped pieces following an extended culture period. These living cell-synthesized tissue pieces (referred to as “sheets” herein) have formed the basis of a method, termed sheet-based tissue engineering (“SBTE”), which enables production of living and completely biological constructs that have high mechanical strengths without the need for any artificial or other exogenous scaffolding.

SBTE has been successfully used to produce human skin, and blood vessels, the latter being currently tested in clinical trials (See, e.g., Michel et al., In Vitro Cell. Dev. Biol.—Anim., 35 (6), 318 (1999); L'Heureux et al., FASEB J., 12 (1), 47 (1998); L'Heureux et al., Nat. Med., 12 (3), 361 (2006); L'Heureux, et al., N. Engl. J. Med., (2007) 357 (14), 1451; U.S. Pat. Nos. 7,112,218 and 7,504,258, all of which are incorporated herein by reference). Some SBTE methods involve stacking or rolling sheet layers and then waiting for them to adhere and strongly fuse together while in culture, to produce thicker tissues. The sheet layers must be maintained in close apposition for an extended period of time, referred to as the maturation period, to achieve fusion of the different layers.

More recently, in a technology termed thread-based tissue engineering (“TBTE”) herein, cell-synthesized threads were produced from a sheet, or directly. The threads can be formed into a fiber to make into various structures such as blood vessels, by using weaving or other techniques from the textile industry (U.S. Pat. App. Pub. No. 2010-0189712, incorporated herein by reference). The resulting completely biological blood vessels can be assembled in days once the threads are produced, thereby avoiding the long maturation period associated with other techniques; they display excellent mechanical properties.

While both SBTE and TBTE produce completely natural, unmodified, mechanically very strong tissue, the resulting constructs are very dense, which is not conducive to the production of thick tissues that need to be perfused by a network of blood vessels to provide adequate exchange of gas, nutrients, and waste. Hence there is a need to develop a natural material, which contains microstructure and micro-organization of tissues similar to the ones found in nature, and will provide a porous structure to build perfusable thick tissues such as liver, kidney, pancreas, brain and adipose tissue. In addition, both sheet-based and thread-based architectures have a limited ability to create complex three-dimensional structures with a wide range of compositional gradients. Finally, SBTE and TBTE are not amenable to creating injectable products.

Other tissue-based materials have been provided in injectable form but have a number of drawbacks. For example, Fascian™ is a dermal filler made from cadaverous tissue, which has cosmetic applications (see, Shore, Ophth. Plastic and Reconst. Surg., 16(1), January 2000). This substance utilizes tissue with a lot of ECM, but comparatively few cells and is therefore not constructed using principles of tissue engineering. Furthermore, Fascian does not utilize viable cells and therefore is usually provided in lyophilized form, or particles that have been milled or shredded. Fascian™ also differs from, and therefore lacks benefits of, tissue-engineered materials because it is not necessarily pure; its origins mean that it can contain all sorts of ground up cells, such as from blood vessels, nerves, and the immune system. This lack of homogeneity means that it is not suitable for a lot of sensitive applications, particularly where rejection may be an issue.

The discussion of the background herein is included to explain the context of the technology. This is not to be taken as an admission that any of the material referred to was published, known, or part of the common general knowledge as at the priority date of any of the claims found appended hereto.

Throughout the description and claims of the specification the word “comprise” and variations thereof, such as “comprising” and “comprises”, is not intended to exclude other additives, components, integers or steps.

### SUMMARY

The instant disclosure addresses the creation and use of cell-synthesized particles. In particular, the disclosure comprises a composition comprising one or more tissue engineered particles, the particles comprising one or more biological cells and an extracellular matrix synthesized by the cells.

The disclosure further comprises a method of making a tissue engineered particle composition, the method comprising: (a) seeding a population of cells in a culture vessel; (b) culturing the cells under conditions that allow the formation of a tissue sheet in contact with an inner surface of the culture vessel, wherein the sheet is comprised of cells and extracellular matrix synthesized by the cells; and (c) cutting the sheet into a plurality of pieces thereby forming a particle composition.

The disclosure further comprises a method of making a tissue engineered construct, the method comprising: (a) placing one or more types of cell-synthesized particle compositions in a container; (b) culturing the particles under conditions that allow the fusion of the particles, thereby forming a tissue; and (c) shaping the tissue into a construct.

The present technology includes a cell-synthesized biological particle, processes for making a cell-synthesized particle, and processes for assembling cell-synthesized particles into tissues and organs. This technology forms the basis of particle-based tissue engineering (“PBTE”, herein). The particle is originally produced on a substrate in vitro from living cells in culture and the extracellular matrix the cells produce. Particles can be comprised of one or more cell types and have various sizes or shapes. Particles, of one or more types, can be used to create more complex constructs that otherwise could not have been generated. The particles are small pieces of tissue, typically less than 2 mm in dimension but no smaller than a few microns, that are engineered from cells and cell-synthesized ECM, and that can be used in various ways. One way is as an injectable method to create, repair, or augment tissue in a localized region of the body. Once injected, surrounding cells migrate to the injected tissue, and help to remodel it and integrate it into the body.

The particles or tissues comprising the particles have mechanical and biological properties that prove beneficial in the medical industry and that can be achieved by altering the climatic conditions during sheet production, varying the stresses and strains on the tissue, or by adjusting the chemical composition of the culture environment. The cell-synthesized particles therefore provide a complementary technology to cell-synthesized sheets and threads; they provide a different form of building block for making tissue-engineered constructs, much as cloth, beads, and rope can be used in textile manufacture.

The processes described herein result in a biological particle that is comprised of unmodified natural ECM. It results in a product that will not cause a significant immune response upon implantation. The particles or tissues can be devitalized or decellularized, either partly or substantially. The particles or tissues can be combined with other components such as biomaterials, chemicals, pharmaceuticals, or biologics, to create new products.

The present disclosure provides for in vivo applications and uses of the cell-synthesized particles described herein.

The technology herein has aesthetic applications, as well as in surgery. For example, the cell-synthesized particles described herein can be injected as dermal fillers for correcting disfiguration, age lines, etc. In this sense, the cell-synthesized particles represent alternatives to existing forms of cosmetic surgery, as well as being complementary to those forms. For example, cell-synthesized particles could be used in conjunction with other dermal fillers made from plastics, ceramics, or cadaverous tissue.

The present disclosure additionally includes an apparatus for culturing tissue in a manner suitable for creating cell-synthesized particles, and an apparatus for cutting particles from a tissue sheet, such as a cell-synthesized tissue sheet.

### BRIEF DESCRIPTION OF THE DRAWINGS

The drawings provide exemplary disclosure that illustrates aspects of the technology herein, when viewed in conjunction with the detailed description of the invention, examples, and claims appended hereto.

FIGS. 1A and 1B show micrographs of exemplary particles as further described herein. Scale bars are shown in each figure. In each case, the particles have been mechanically conditioned by bunching up, for example with tweezers, the fragments of tissue originally cut from a tissue sheet. FIG. 1A is a bright field micrograph of dried particles of various sizes, approximately 75 μm, 150 μm, 250 μm and 350 μm in diameter. FIG. 1B is a phase contrast micrograph of the same three smallest particles in FIG. 1A, after hydration. The approximate sizes are now 165 μm, 260 μm and 600 μm, showing that the particles' volumes have significantly increased after hydration. Although the particles can be stored dry, they will normally be used in a ‘wet’, or hydrated, form. Hydration can occur relatively easily, for example by immersing a particle in an aqueous medium. A typical particle will hold about 50% of its dry weight in water, when hydrated.

FIG. 2 shows a phase contrast micrograph of hydrated particles that were cut to produce particles that are more angular in shape than those in FIGS. 1A and 1B. These particles were not mechanically conditioned to produce more rounded particles. The particles of FIG. 2 were produced from a tissue sheet 180 micron thick, which is thicker than the sheet used to generate the particles of FIGS. 1A and 1B.

FIG. 3 provides a graph showing how particles remodel after creation depending on whether they comprise living or dead cells. The remodeling is evidenced by tissue contraction, here measured by a change in two-dimensional surface area (about 50% decrease by day one), due to the activity of live cells (left-hand (dark) bars at each time-point). If the cells are killed prior to forming the particles, the projected surface areas of the particles remain constant at about 1.4 mm² (right-hand (light) bars at each time-point).

FIG. 4 shows a rod-shaped fragment of tissue created by particle-based tissue engineering, as further described herein. Particles were produced from a human fibroblast-synthesized sheet, further seeded with human endothelial cells, and assembled in a tubular mold with a porous bottom. The tissue was cultured for 5 days in a centrifugal bioreactor to perfuse media into the tissue, before unmolding. The rod of tissue is about 5 mm thick and can be handled with tweezers even at this early stage in its maturation.

FIG. 5 shows histological sections of a tissue obtained by particle-based tissue engineering after 5 days in culture under centrifugal perfusion. Hematoxylin and eosin stain (panel A) shows the fused particles and a complex network of channels of various sizes. Living fibroblasts are distributed throughout the tissue. Masson's trichrome stain (panel B) highlights the high collagen content of the tissue.

FIG. 6 shows seeding of devitalized (A) and living (B) particles created by particle-based tissue engineering, with endothelial cells. Both stains highlight the coverage of the fibroblast-synthesized particles with a secondary cell type, here endothelial cells. Panel A shows immuno-labeling of endothelial cells seeded at the surface of particles that have been previously devitalized, and cultured for 5 days. The antibody used is directed against VE-cadherin, which is a molecule specific to endothelial cells and located at the cell-cell interface. In addition, cell nuclei are stained using Hoechst 33342. Panel B shows immuno-labeling of endothelial cells seeded at the surface of living particles and cultured for 5 days. The antibody used is directed against von Willebrand factor, which is a molecule specific to endothelial cells and located in the cytoplasm of the cells. In B, since the particles contained live fibroblasts, a tissue formed over the 5 days of culture as evidenced by the visible channels. In contrast, the devitalized particles in panel A did not form a cohesive tissue and fell apart when handled.

FIGS. 7A and 7B show an exemplary cutting apparatus for creating cell-synthesized particles from a tissue sheet, in overview (7A), and in a close-up view of the cutting portion (7B).

### DETAILED DESCRIPTION

Disclosed herein are cell-synthesized biological particles for the repair of damaged or defective organs or tissues, as well as apparatus, materials and methods relating to the production and use of the particles, and compositions thereof. The cell-synthesized particles can be created using techniques of tissue engineering. The cell-synthesized particles can also be used as in vitro models for research purposes. The cell-synthesized particles can be assembled into complex three-dimensional constructs for use as cell culture scaffolding, as tissue or organ replacement; they can be used as bulking agents—for example as part of an injectable composition, as drug delivery systems, or as wound healing devices.

### Creating Cell-Synthesized Particles

In one embodiment of the technology described herein, cell-synthesized particles are cut from a tissue sheet. The tissue sheet is preferably one constructed from other methods described or referenced elsewhere herein, and comprises living cells and extracellular matrix produced by the cells. It has high mechanical strength without the need for artificial or other exogenous scaffolding. Suitable tissue sheets can be 50 to 300 μm thick, and typically about 50-100 μm thick. Exemplary methods and apparatus for creating such sheets are methods of sheet-based tissue engineering, as described in Michel et al., In Vitro Cell. Dev. Biol. -Anim., 35 (6), 318 (1999); L'Heureux et al., FASEB J., 12 (1), 47 (1998); L'Heureux et al., Nat. Med., 12 (3), 361 (2006); L'Heureux, N., et al., N. Engl. J. Med., 357 (14) (2007), 1451; U.S. Pat. Nos. 7,112,218 and 7,504,258, all of which are incorporated herein by reference.

Tissue sheets can be produced by seeding fibroblasts on a sterile substrate that supports cell proliferation at a density of about 1×10⁶ cells/cm² or less, preferably about 100 cells/cm² to 1×10⁵ cells/cm², more preferably between about 1×10³ cells/cm² to 5×10⁴ cells/cm², most preferably about 1×10⁴ cells/cm². Cells are preferably grown on a sterile culture medium that supports cell proliferation and ECM production. An example of such media is a base 3:1 mixture of Dulbecco's Modified Eagle's Medium (DMEM) (high glucose formulation, without L-glutamine) and Hams F-12 medium supplemented with either 4 mM L-glutamine or equivalent, 20% Fetal Bovine Serum (preferably immunoglobulin-depleted) and 50 μg/ml sodium ascorbate, or other collagen synthesis promoting agents. Other additives can be used to promote collagen or other ECM components (e.g., elastin) production. Alternatively, a serum-free medium can be used; this media can be chemically defined. Growth media is completely or partly replaced with fresh media every 2 to 5 days. Cells are maintained in an environment containing about 10% CO₂, about 20% O₂, and at a temperature of about 37° C. Sheets can also be grown in low-O₂ concentration or in media that do not require CO₂. Other cell-compatible reagents and culture conditions that support cell proliferation and ECM production may be ascertained by one skilled in the art of mammalian cell culture. Typical periods of culture for the sheets are about 24 weeks or less, preferably about 1 to 16 weeks, more preferably between about 4 and 12 weeks, most preferably about 8 weeks.

The cell-synthesized particles can be created by cutting a tissue engineered sheet into very small pieces having all sorts of shapes but typically rectangles. These rectangles can be formed in a range of different sizes (for example, 0.01 mm² to 400 mm² in area, corresponding to dimensions of 0.1×0.1 mm up to 20×20 mm) in order to create a wide variety of particle aggregate compositions. In this embodiment, the particles have one dimension that corresponds to the thickness of the sheet. In addition to rectangles, other shapes (square, circle, oval, triangle, etc.) could be cut from the sheet to produce even more complex constructs, to improve ease of manufacturing or to improve therapeutic use and effect. Cutting patterns can produce different shapes and sizes from the same sheet. In one embodiment, a sheet is cut into particles of the same shape and size as one another. In another embodiment, particles of different shapes and/or sizes are cut from the same sheet. It is to be understood that, when describing a width or a diameter of a particle, it is not implied either that the particle is circular in one cross-section, or even uniform in cross-section. A width or a diameter can therefore refer to an average dimension for that particle, or a measurement of a largest distance between two points on a perimeter of a particle.

Cutting of the sheet can be achieved by means of one or more mechanical devices such as a blade, a cutting die, a cutting wheel, a punch or a needle. Breaking the sheet into particles can also be performed using light (including lasers), sound, thermal or other forms of energy. Cutting can also be performed with a jet of liquid or particulate. In order to achieve greater precision, the cutting can use a mask, a guide or be computer controlled or assisted. The cutting process(es) can be partly or completely automated.

In another embodiment, cutting of particles from a tissue sheet can be achieved with use of a hollow needle or an array of hollow needles, each of which punches a particle out from the sheet. In general, such a method needs a high force to be applied to the needles. Needles of this description can create particles of size 50 micron by 50 micron, but other sizes are possible, such as in the range 10-100 micron by 10-100 micron. It is also usually necessary to force the particles out from the opening in the needle, for example, by sending a jet or puff of air down the needle.

In another embodiment, particles can be created from a tissue sheet using a custom-designed shredder 1, as shown in FIG. 7A. The precise appearance of such a shredder is shown in purely exemplary form in FIGS. 7A and 7B. Central portion 3 of shredder 1 contains a tissue sheet (not shown). The sheet can be held taught between upper plate 7 and lower plate 5. Tension of the sheet can be adjusted by one or more screws 9. A cutting unit 15 comprises an array of rods 11. The rods in the embodiment shown are arranged in two parallel rows, wherein the positions of the rods in the two rows are offset with respect to each other. Each rod contains a cut-out 13 (shown in enlarged view in FIG. 7B) that is configured to act as a blade, so as to apply a cutting action to the tissue sheet. In the embodiment shown, the cutting unit advances in the direction of the arrow, and works down the sheet creating a number of particles as each row of rods intersects the leading edge of the sheet. The rods can facilitate cutting by additionally moving vertically (perpendicular to the plane of the sheet) when they contact the edge of the sheet. A fluid stream or a brush can be used to dislodges the particles from the cutter so that they fall into a receptacle (not shown) situated beneath the sheet. Rods of a range of sizes can be used, in order to obtain particles of different sizes, consistent with other descriptions herein.

The cutting can be performed at any stage of the sheet formation and may be done before, during or after detachment of the sheet for the substrate it was grown on. If the sheet is cut while still attached to the substrate, the detachment of the cell-synthesized particles can be achieved mechanically, enzymatically, chemically, by thermodynamic changes (such as application of temperature or pressure changes), or using other methods known in the art. The sheet can be dehydrated or devitalized prior to creating the particles. In one embodiment, the cutting pattern can result in perforated lines that allow the detachment of the particle at a later time. The cutting pattern can leave very small segment of tissues connecting the particles to other particles, or to the remaining part of the sheet. This is a way of creating strings or groups of particles as well as sheets with borders of particles.

Particles can also be produced without cutting from sheets if clusters of tissue are grown on very small discrete culture surfaces to produce particles directly. Discrete surfaces can be created by selectively treating regions of the culture substrate to promote cell adhesion on an otherwise non-permissive substrate or, alternatively, by treating defined zones to inhibit cell adhesion on a generally permissive substrate. Both approaches can be used in combination. These surfaces can have a multitude of shapes, can share the same culture media, and can be connected by thin regions to allow for particles to be separated, or broken apart, at a later stage. The particles can be detached from the substrate using methods described elsewhere herein.

Cell-synthesized particles as described herein differ significantly from cellular aggregates previously described in the scientific literature (for example, Kelm, J. M., et al., J. Biotechnol., 17 (2010); and Napolitano, A. P., et al., Tissue Eng., 13 (8), 2087 (2007)). Often referred to as “self-assembled”, these cellular aggregates are created from single cell suspensions, or small groups, that are prevented from adhering to a substrate. These aggregates are composed principally of cells and do not contain significant amounts of ECM. This difference has important consequences for the subsequent assembly of tissues from the aggregates and for possible therapeutic uses. The presence of ECM positively influences the cell behavior and survival, and provides mechanical structure. In addition, cell aggregates created in suspension can have excessively high, non-physiological, cellular densities, which will typically lead to cell death in culture or upon implantation. The lack of ECM and the excessive cell densities would require significant remodeling of a construct made from such aggregates, upon implantation. Furthermore, excessive amounts of dead cells and cell debris can cause excessive inflammation. As a result, the geometry of an implant made from cellular aggregates can change with time in a manner that may be negative.

By contrast, because of their complex but natural ECM, and because their cellular densities are more typical of normal tissues, the cell-synthesized particles described herein can allow manufactured tissues and organs to better maintain their structure once implanted.

### Sheet and Initial Particles

The particles can be produced from autologous or non-autologous (allogeneic) human cells, from animal (xenogeneic) cells, from genetically modified cells (human or animal) or any combination thereof. The particles can be formed completely from human cells, and even autologously to avoid immune reactions.

While fibroblasts are the preferred cell types for particle production, particles can be produced using any other ECM-producing cell type such as, but not limited to: a variety of so-called stromal cells, myofibroblasts, myocytes or their precursors, smooth muscle cells or their precursors, macrophages, osteoblasts or their precursors, mesenchymal stem cells, bone marrow-derived stem cells, fat-derived stem cells, skin-derived stem cells (including appendages), embryonic stem cells, induced pluripotent stem cells or any combination thereof. These cells can be seeded at the same time or sequentially during sheet production. Certain cells may have key advantages in terms of immunogenicity, but the methods and application of particle-based tissue engineering as described herein are not limited to particular cell type(s). In some embodiments, the cells are not fibroblasts, and the cell culture medium does not include fibroblasts.

In addition, non-ECM producing cells can be incorporated with ECM-producing cells to perform other biological functions, to facilitate manufacturing or to serve a therapeutic role upon implantation. These cells can be added at the beginning of, or during, the production of the tissue, for example, a sheet. Alternatively, non-ECM producing cells can be seeded on the surface and/or bottom of the sheet at the end of the sheet production process (prior to particle formation). Non-ECM producing cell types that can be used in this way include, but are not limited to, neurons or their precursors, glial cells or their precursors, islet cells or their precursors, hepatocytes or their precursors, endothelial cells or their precursors, mesothelial cells, keratinocytes, or cells from the nephron.

The substrate used to produce the sheet can be a film or thin layer of biocompatible material such as, but not limited to, a permanent (such as non-biodegradable) synthetic polymer (e.g., ePTFE), a resorbable or biodegradable synthetic polymer (e.g., polyglycolic acid), a biological material (e.g., collagen, elastin, proteins, peptides). In one embodiment, the substrate can be cut with the sheet and become part of the particle, thereby producing a composite particle. Such composite products provide an easier way to manufacture particles by avoiding the step of separating the biological material from the substrate. In some circumstances, the substrate itself may be beneficial to the healing of the cell-synthesized particles after they have been injected into a patient, or molded as further described herein. The substrate could also be a filler that reduces the costs of the product per volume unit.

During sheet growth, exogenous components can be added to the culture medium and become embedded or otherwise incorporated into the sheet, and eventually become part of the particles produced from that sheet. These components include, but are not limited to, natural or synthetic peptides, proteins, glycoproteins, proteoglycans, antibodies, polysaccharides, DNA or RNA, transfection agents, antibiotics, pharmaceutical agents, metal particles, insoluble mineral crystals or particles, polymer particles, radioactive agents, drug delivery systems, or radio-frequency identification (RFID) chips or other magnetic or electronic devices. Besides identification purposes, these additives can serve to facilitate or enhance further manipulation, storage, diagnostic, surgical use or healing.

Post-Production Treatment of the Particles

The cell-synthesized particles can be living to provide a desirable biological effect or advantageous healing, when introduced into a subject. However, the sheet used to produce the cell-synthesized particles, or the particles themselves, can also be devitalized or decellularized (both processes will be referred to by the term “devitalization” elsewhere herein). The devitalization can be complete or partial. It can be achieved by any, or a combination of, methods known in the art including, but not limited to, drying, heating, cooling, freezing, adding chemicals, such as acids, alkalis, enzymes, salts, toxins or solvents, or applying various forms of energy, including, but not limited to ultrasound, radiation, mechanical forces and osmotic pressure. The devitalization process may have secondary effects and be performed for ulterior purposes, such as increasing mechanical strength, reducing immunogenic or thrombogenic properties, facilitating manufacturing, or improving the chances of implantation success. This could improve the healing effect of the particles by, for example, reducing the risk of adverse immune reactions in allogeneic or xenogeneic implantation. This could also simplify the processing, storage and shipping of the particles. Decellularization may also be necessary or practical for the assembly of more complex particle-based products.

After detachment from the culture substrate, the cell-synthesized particles can be used directly for implantation or tissue assembly, or they can be maintained alive in culture to allow tissue contraction or other biological changes to occur. The particles can be mechanically conditioned under stress produced by forces such as those associated with stirring, compressing, or rolling. FIG. 4 shows how particle surface area changes over time in culture, depending on whether the cells are dead or alive.

In one embodiment, particles are maintained in suspension in a stirring bioreactor. Inducing fluid shear stress on the particles can be particularly important in maintaining cell viability thereby producing a useful and beneficial product. It is understood that this may be because the action of stirring the liquid medium accelerates the transfer of O₂ into the cells. It is also well-known that cells sheared with liquid change their expression profile. The action can also induce cells to produce or degrade collagen or other proteins. Living (non-devitalized) cell-synthesized particles can be stored in a bioreactor or any other storage system known in the art to apply mechanical stresses and strains (mechanical conditioning), or establish other environmental conditions (such as appropriate O₂ concentration and temperature), while maintaining viability of the particles. Cell-synthesized particles can also be cryogenically preserved and stored in frozen form using methods known in the art to maintain viability. It is understood that it is almost impossible to achieve complete (total) viability of all the cells; some cells always die.

The particles can also be mechanically processed to shape them into more spheroid or ovoid particles by, for example, rolling the particles on a surface. This can be done in conjunction with one or more drying or rehydration steps. This can provide, among other things, better injectability characteristics to the particle preparation.

In addition or alternatively, the cell-synthesized particles or the sheet can be treated with powerful cross-linking agents of various strengths, including aldehydes, which can be used to achieve a complete or partial devitalization, to reduce immunogenic effects, to retard biodegradation, to modify mechanical properties or simply to attach chemical or biological compounds to the particles or sheet directly.

A particle, as described elsewhere herein, can also be devitalized and/or cross-linked under conditions such as the foregoing. A living particle can be devitalized at any of the production steps described herein. During or after the devitalization process, the living particle can be mechanically conditioned in any or in multiple directions by compressive, magnetic, isometric, isotonic, modulated or other types or regiments of forces including fluid shear stress. The devitalized particle can be stored in various liquids such as aqueous solutions, alcohol, or solutions of cross-linking agent, and dried or frozen.

At any time during the production of the cell-synthesized particle, the living or devitalized particle, or the sheet used to create the particle, the product can be coated with various agents to improve healing, mechanical properties or therapeutic effect, to label the product, or to facilitate further manufacturing. These agents include, but are not limited to, exogenous proteins or peptides, proteoglycans, glycosaminoglycans, natural deoxy-ribonucleic acid (“DNA”), recombinant DNA, natural ribonucleic acid (“RNA”), recombinant RNA, viral agents, transfection agents, growth factors, anti-proliferation agents, antibodies, antibiotics or antiseptics or any fragment or combination of these compounds.

Before, after or at any time during a process of making a particle, the living or devitalized particle, or the sheet used to create the cell-synthesized particle, can be seeded with new, or additional, cells. The additional cells can be of the same or of a different type than the ones used for the particle production, or a combination of cell populations. These additional cells can be autologous or non-autologous human (allogeneic) cells, animal cells, genetically modified cells (human or animal) or any combination thereof. The additional cells can be ECM-producing, or non-ECM producing cells. The additional cells can be used to improve healing, mechanical properties or therapeutic effect, to label the product, or to facilitate further manufacturing. The result of using additional cells, in effect, forms a composite particle, which can be altered in any, or all of, the ways previously described.

Adding specialized new cells to the particles can be a tool to repair or replace target tissues and organs. For example, in order to produce new kidney tissue, primary renal cell populations can be seeded on the surface of the particles prior to injection. Once the renal cells have attached to the particles and sufficiently proliferated, which can be achieved in an adequate bioreactor, the cell-seeded particles can be injected in an appropriate location to create the new tissue. Alternatively, the specialized cells can be added to the sheet prior to cutting the particles, or can simply be co-injected with the particles. If the presence of fibroblast has a negative effect on the outcome, devitalized particles can be used instead.

In another example of adding additional cells, cardiomyocytes, myocytes or their precursors can be added to the particles and, possibly, endothelial cells, and injected in the wall of a failing heart. While cell-based therapies of a failing heart have shown some promise, a vexing problem is the very low retention and survival rate of cells injected in the wall of the heart. Injecting cells attached to particles could increase retention due to simple mechanical hindrance, and could increase survival due to the fact that cells are attached to a natural ECM and not in suspension.

A principal application of the cell-synthesized particles described herein, is as part of an injectable composition. Such a composition can be injected directly into a site where repair is needed. The gauge of needle used for injection will vary according to the average size of the particles. A composition containing the particles herein could therefore find application as a dermal filler, for example, in cosmetic surgery. Such a composition differs from non-living and inorganic materials (e.g., plastics and ceramics) used in the art, at least because it offers the possibility of becoming truly integrated into the surrounding tissue. As described elsewhere herein, the tissue-engineered particles of the present technology also differ from Fascian, a dermal filler made from cadaverous tissue, which has cosmetic applications in that they offer better quality control, because they can for example be produced from a single cell-line/donor.

Assembly of Cell-Synthesized Particles into Complex Structures

Cohesive and completely biological living tissues with porous structures and complex geometries can be produced from the living cell-synthesized particles. These tissues are not as mechanically strong as tissue created with SBTE or TBTE, because of the significant void space within the tissues. Strength is dependent largely on tissue fusion, and since there are voids, only part of the particles will be in contact. Sheets made by SBTE have intrinsic strength, and threads made by TBTE have arrangements that produce strength without fusion.

However, the particles created by the approaches described herein have other key advantages over tissue sheets and threads. Most notably, PBTE produces tissue with a large void space, which allows for the development of an intricate network of channels. These channels can be used to create blood vessels, lymphatic vessels, secretory ducts, airways or other similar structures. For that purpose, specialized cells can be seeded in the void space. These structures can mimic various organs and tissues such as the liver, kidney, brain, pancreas, lung, adipose tissue, bone, cartilage or others. The void space in these constructs can also be filled with other materials such as exogenous ECM (ECM that has been sourced elsewhere, and contains, for example, fibrin, or elastin), to achieve the desired structure or therapeutic effect. In addition, PBTE can facilitate the production of complex geometries because of the wide range and fine distribution of geometry, porosity and cell content that can be achieved. For example, by assembling particles of various sizes and of various shapes, a wide array of tissue porosity can be created. Taken together, these characteristics mean that PBTE provides significant advantages for the production of vascularized bulky organs. Taking advantage of the various assembly techniques that are available, bulky organs can be assembled with a network of pores or channels that can be connected to the body's vasculature.

The cell-synthesized particles can be used to produce various two-dimensional (i.e., flat) but preferably three-dimensional constructs. In the simplest embodiment of the invention, living particles in suspension, in a culture medium in a bioreactor, are allowed to settle to the bottom of the container and maintained in environmental conditions favorable for cell survival. After a maturation period that ranges from hours, to days, to weeks, and even to months, particles will fuse together as a result of cellular activity (for example, cell-cell adhesion, cell-ECM adhesion and ECM synthesis). The maturation time is typically controlled by the developer, and is decided based on experience with specific cell types and the goal (defined by a parameter such as tissue hardness) for a particular application.

Methods to induce particle fusion can include various physical strategies (centrifugation, compression, concentration, magnetically loading the particles by, for example, including a magnetic substance in the particles and then applying a magnetic field to exert force on the particles, electrostatic forces, etc.). In one embodiment, particles are placed into a sterile tube with one open end and the other end sealed off with filter paper. The particle filled tube is placed into a medium filled bioreactor and allowed to spin concentrically to induce both centrifugal forces and medium flow through the particle mass. Pharmacological methods of inducing particle fusion can also be used: one way is by introducing additives that promote cell activity into the medium. Adhesive agents such as biological or chemical glues can also be used. Molecular adhesives such as ligand-receptor combinations using peptides, proteins, antibodies, etc., can be used. Enzymatic activity from exogenous sources can also be used. In another embodiment, the particles are embedded in a hydrogel or other compound that creates a cohesive tissue containing multiple particles. In another embodiment, particles are enclosed in a biocompatible membrane or other device forming an enclosure. In another embodiment, devitalized particles are used and a chemical crosslinking agent is used to create inter-particle attachment.

The constructs can be produced by successive assembly of particles to create layers with the same or different properties. Constructs can be produced by combining both living and devitalized particles of the same or differing cell types (any particle composition can be used). Particles of different sizes can be used. The geometry of the settling surface can be varied to control construct geometry. Particles can be assembled in a mold or a cast. By using specific timing and specially tailored distributions of particle sizes, a wide range of complex structures can be produced.

Additional void space in a tissue construct can be created by completely or partly embedding an exogenous object during particle assembly, and then removing this object once the particles are fused. Alternatively, the exogenous object can be left in the assembly to improve particle fusion, mechanical properties, healing or therapeutic effect, to label the product, or to facilitate further manufacturing. The object can be made of materials that can be natural or synthetic and permanent or resorbable in nature. The object could be any of the exogenous components mentioned herein as possibly embedded or otherwise comprised in the tissue sheet from which the particles are cut. In addition, the object can be physically larger than those embedded in sheets, since the assembled particle-based construct can be much larger than the thickness of a sheet. Additional objects could include tubes or cannulas and various types of connectors, handles, wires, loops, magnets and connecting, suturing or anchoring devices to facilitate tissue manipulation, implantation or further manufacturing. These objects could also include devices such as cardiovascular stents or vascular grafts.

These constructs can undergo the same treatments and procedures, and for the same reasons, as previously described for individual particles. For example, the constructs can be maintained alive to allow further cellular activity and remodeling. They could be stored in various liquids, dried or frozen. The living construct can be frozen using methods known in the art to maintain partial viability. The construct can be partially or completely devitalized, exposed to mechanical forces, cross-linked or coated with various agents, as described elsewhere herein.

After, or at any time during, the construct production, new cells can be added to the construct. The cells can be of the same or of a different type than the ones used for the particle production, or a combination of cell populations. These cells can be autologous or non-autologous human cells (allogeneic), animal cells, genetically modified cells (human or animal) or any combination thereof. These cells can be ECM-producing or non-ECM-producing cells. These additional cells can be used to improve particle fusion, mechanical properties, healing or therapeutic effect, to label the product, or to facilitate further manufacturing. Channels in the construct can be seeded with the appropriate specialized cells to mimic the tissue or organ to be replaced.

More complex constructs can be produced by combining cell-synthesized particles as described herein with cell-synthesized sheets (see for example, U.S. Pat. Nos. 7,112,218 and 7,504,258) and cell-synthesized threads (U.S. Pat. Application Publication No. 2010-0189712), all of which are incorporated herein by reference in their entireties.

Particles as described herein can be used to provide supporting structures for organ repair, as well as replacement of almost any bodily tissue, and used in applications including but not limited to, repair or replacement of the liver, kidney, brain, pancreas, lung, adipose tissue, bone, and cartilage. In addition, particles can be used for plastic and reconstructive surgery as regenerative, filling or bulking agents. In one embodiment, the particles can be injected with a syringe-like device.

Tissue constructs can be assembled by culturing cell-synthesized particles in vitro. For example, particles can be settled into a mold, such as by placing one or more types of cell-synthesized particles into a container, and combined with other media. Other cells can be introduced to help the particles fuse together and form the tissue. The molded tissue can then be shaped into the desired construct, for example by using various shaping tools.

The particles as described herein can be made in a range of sizes. For example, where a particle is cut from a sheet of cells, the particles may have a width ranging from 100 microns to 2 cm. Other ranges of particle width include 10-150 micron, 1 mm to 10 cm, 0.5 cm to 5 cm, and 1 to 2 cm. Still other ranges include: 10-25 micron; 25-50 micron; 50-75 micron; 75-100 micron; 100-150 micron; 150-200 micron; 200-250 micron; 250-300 micron; 300-500 micron; 500-750 micron; 750-1000 micron; 1000-1250 micron; 1250-1500 micron; 1500-2000 micron; 2000-2500 micron; and 2500-5000 micron. It is to be understood that other ranges composed of a lower endpoint and an upper endpoint selected from different respective ranges of the foregoing list of ranges could also be used to describe a useful range of particle widths. It is further to be understood that ranges expressed as exact numbers (e.g., 250 micron) could alternatively be expressed as having some imprecision, (e.g., “about 250 micron”), thereby encompassing values falling slightly outside of the exactly stated ranges. Typically, the use of “about” could represent an imprecision of +1-5%. Other exemplary widths are 0.4, 0.6, and 0.8 cm. A preferred particle size falls in the range 0.1-1 mm. Sizes can be expressed as averages over an ensemble. So that, for example, an ensemble of particles cut from a sheet can have sizes within a particular range, e.g., 0.3-0.5 mm in length and width, i.e., the average over the ensemble is from 0.3-0.5 mm. As described elsewhere herein, a thickness of a particle derives from the thickness of the sheet from which it is cut and can typically be 0.05-0.3 mm. Expression of particle sizes by linear dimensions does not preclude other ways of quantifying size, such as by stating a corresponding volume.

### EXAMPLES

### Example 1

### Injectable Cell-Synthesized Particles

A cell-synthesized tissue sheet (produced by methods described elsewhere herein) is cut to produce particles after a period of culture of 24 weeks. For this application, particles of about 1 mm² in area are preferred, which approximate the volume of a spheroid of about 250 μm in diameter, but any particle size that results in an injectable particle can be used. The sheet is detached mechanically from the culture substrate and transferred to cutting boards. Using a multi-bladed instrument, the sheet is cut in one direction and then in a perpendicular direction to create separate particles. The particles are put in suspension in a cell-compatible liquid to create cell-synthesized particles. The particles can be injected immediately or allowed to retract into a more spherical shape or otherwise remodelled for a period of time that can range from a few minutes to many months. This can be achieved in a bioreactor that provides specific culture conditions, such as stirring or the media, which promotes particle remodeling and survival to improve therapeutic effect.

Before implantation, particles can be rinsed, for minutes to days, with a specific buffer to remove undesirable components of the culture media, such as bovine serum, that could adversely affect a successful implantation outcome. They can also be incubated with, or simply co-injected with, an agent that promotes healing of the implant, adds volume to the preparation, or has other desirable effects. For example, an agent can promote vascularization at the site of injection to improve particle survival and neo-tissue creation and stability. In another embodiment, the agent favors calcification if the injected particles are intended as a bone repair treatment. In another embodiment, the particles are injected with fibrin glue, or other scaffolding gel, to form a more cohesive mass after injection.

### Example 2

### Porous Scaffolds for Tissue Repair

In this embodiment, living particles are produced (1 mm² cross-sectional area) and maintained in a bioreactor in culture media under stirring conditions (40 RPM) for 3 days at 37° C. and 10% CO₂. Particles are then cast in a mold with a porous bottom. For example, the mold can be a silicone cylinder (1 cm internal diameter) with one extremity closed by a mesh of polyester with a 100 μm pore size. Once particles are poured in the tube, the other extremity can be closed with an insert that has a central opening of 1 mm internal diameter. The mold is then mounted on a rotating shaft so that the mesh is facing away from the shaft and the insert is facing the shaft (the tube is oriented horizontally while the shaft is vertical). As the shaft rotates around its longitudinal axis, the tube is oriented radially to the shaft. The resulting centrifugal force maintains the tissue against the mesh and insures media exchange within the tissue. After 3 weeks, the particles have fused into a tissue that can be removed from the mold. This tissue can be implanted in the patient to fill a defect. The void space in the tissue will favor vascularization of the graft and maintenance of the overall geometry.

In an alternative approach, the tissue is devitalized and entirely repopulated with the patient's own cells. This approach has significant advantages from the manufacturing point of view since a devitalized graft can be stored and shipped much more easily than a living tissue. In addition, a devitalized tissue may lead to enhanced healing if the sheet was produced with allogeneic cells by avoiding some immune reaction. Even a devitalized autologous tissue may lead to better outcomes if fibroblasts inhibit vascularization of the tissue or other benefic remodeling. Better healing can also be achieved by adding, for example, a heparin coating bound to the luminal surface of the void space of the construct. This heparin can further be loaded with vascular endothelial growth factor that will accelerate vascularization of the matrix.

One cosmetic application would be to sculpt contour deformities or replace oncologic defects. Numerous reconstructive, cosmetic, and correctional possibilities exist for the development of a clinically translatable strategy with which to restore a volume of removed cancerous tissue with the patient's own vascularized tissue.

### Example 3

### Porous Scaffolds Containing Specialized Cells

As described elsewhere herein, a living or devitalized cell-synthesized particle can be used to create a porous scaffold. The scaffold can be seeded with a cell population before implantation. This seeded scaffold would have the capacity to mimic or supplement normal function of any organ based on the cell population. This cellular mimicry could be used to perform a biochemical role, for example the production of insulin, or a structural role. In one embodiment, endothelial cells or their precursors could be seeded to create a functional network of blood vessels more rapidly than through spontaneous remodeling where endothelial cells migrate from the recipient surrounding tissue. By having a functional vasculature, the implant is more likely to maintain its original shape and provide a better product for cosmetic and reconstructive surgery. During the formation of the tissue, one or more tubular structures can be partly integrated in the tissue so that a free end remains to be grafted to blood vessels of the recipient, and the other end is embedded in the tissue and connected to the channels in the tissue, hence allowing for perfusion of the tissue. This perfusion can also be performed in vitro to improve tissue formation and prepare for implantation.

In addition, specialized cells can be used with endothelial cells to added additional functions. For example, if seeded with adipocytes or their progenitors, the particle construct could provide surgeons with a source of patient-specific adipose tissue of a predefined volume for breast reconstruction. If seeded with pancreatic cells, this construct could produce insulin when challenged with glucose. If seeded with primary renal cells, this construct could reorganized in a functional blood-filtering unit. If seeded with hepatocytes, this construct could be grafted to a failing liver to enhance detoxification functions.

### Example 4

### Research Model

Particles can be used for the construction of complex cell culture models that recreate aspects of the in vivo tumor microenvironment to study the dynamics of tumor development, progression, and therapy on multiple scales. Particles can be used for the construction of complex cell culture models to study vascular network development under perfusion.

### Example 5

### Combination of PBTE, TBTE and SBTE

Cell-synthesized sheets, threads and particles each have clear and distinct advantages when it comes to tissue and organ designs. In this embodiment, an enclosure can be created by wrapping a sheet upon itself. This enclosure can be completely or partially sealed by using threads as a suture material and sewing the edges of the sheet together. The enclosure can then be filled with particles seeded with specialized cells (e.g., hepatocytes). The enclosure can be connected to tubes created from sheets, as described in U.S. Pat. 7,112,218, to create a perfusion system. The void space of this tissue can be seeded with endothelial cells. After an appropriate in vitro culture period, this assembly can be implanted in a patient by connecting the tubes to the circulatory system of the patient. Some tubes could be connected to the hepatic duct, the bile duct, or directly to the duodenum. Such constructs can provide a completely biological liver-like structure. The sheets and threads can be replaced by biocompatible materials. One skilled in the art can see that any number of organs can be created by varying the specialized cells used, the geometry of the construct and the implant location. This approach can even be used to create a lung by lining the interior surfaces with endothelial cells and airway epithelium and by connecting some of the tubing to a bronchus.

It is to be understood that although the methods and apparatus described herein are typically applied to create tissue particles for use in humans, the methods and apparatus could also be recruited to create tissue particles for veterinary and agricultural applications, such as for pets and farm animals. Typically the methods and apparatus herein will be applied to mammals, though such methods could also be used to create tissue for use in birds, reptiles, amphibians, and aquatic organisms such as fish.

The foregoing description is intended to illustrate various aspects of the instant technology. It is not intended that the examples presented herein limit the scope of the appended claims. The invention now being fully described, it will be apparent to one of ordinary skill in the art that many changes and modifications can be made thereto without departing from the spirit or scope of the appended claims.

All references cited herein are incorporated herein by reference in their respective entireties and for all purposes.

What is claimed:

1. A composition comprising one or more tissue engineered particles, the particles comprising one or more biological cells and an extracellular matrix synthesized by the cells.

2. The composition of claim 1, wherein the biological cells comprise two or more cell types.

3. The composition of claim 1, wherein the cells have been substantially devitalized.

4. The composition of claim 1, wherein the cells have been genetically engineered.

5. The composition of claim 1, wherein the particle has a diameter from about 50 micrometers to about 5 mm.

6. The composition of claim 1, further comprising an exogenous component.

7. The composition of claim 6, wherein the exogenous component is a natural or synthetic constituent selected from: peptides, proteins, glycoproteins, proteoglycans, antibodies, polysaccharides, DNA or RNA, transfection agents, antibiotics, pharmaceutical agents, metal particles, insoluble mineral crystals or particles, polymer particles, radioactive agents, drug delivery systems, or radio-frequency identification chips or other magnetic or electronic devices.

8. A method of making a tissue engineered particle composition, the method comprising:
(a) seeding a population of cells in a culture vessel;
(b) culturing the cells under conditions that allow the formation of a tissue sheet in contact with an inner surface of the culture vessel, wherein the sheet is comprised of cells and extracellular matrix synthesized by the cells; and
(c) cutting the sheet into a plurality of pieces thereby forming a particle composition.

9. The method of claim 8, wherein the cells comprise one or more types of cell selected from: fibroblasts, stromal cells, myofibroblasts, myocytes or their precursors, smooth muscle cells or their precursors, macrophages, osteoblasts or their precursors, mesenchymal stem cells, bone marrow-derived stem cells, fat-derived stem cells, skin-derived stem cells (including appendages), embryonic stem cells, induced pluripotent stem cells or a combination thereof.

10. The method of claim 8, wherein the population of cells has been genetically engineered.

11. The method of claim 8, further comprising detaching the plurality of pieces from the substrate.

12. The method of claim 8, further comprising removing the tissue sheet from the substrate before the cutting.

13. The method of claim 8, wherein the area of each of the pieces is from about 0.01 mm² to about 400 mm².

14. The method of claim 8, further comprising substantially devitalizing the tissue sheet or the biocompatible particle composition.

15. The method of claim 8, further comprising:
(i) culturing the adherent cells in the presence an exogenous component; or
(ii) associating an exogenous component with the tissue sheet or the formed particle composition.

16. The method of claim 8, further comprising treating the tissue sheet or the particle composition with a cross-linking agent.

17. The method of claim 8, further comprising drying the tissue sheet before cutting.

18. A method of making a tissue-engineered construct, the method comprising:
(a) placing one or more types of cell-synthesized particle compositions in a container;
(b) culturing the particles under conditions that allow the fusion of the particles, thereby forming a tissue; and
(c) shaping the tissue into a construct.

19. The method of claim 18, further comprising partially devitalizing the construct.

20. The method of claim 18, further comprising seeding one or more cells in or on the tissue.

21. The method of claim 18, wherein the cell-synthesized particles are made by the method of claim 8.

22. The composition of claim 1 in a form suitable for injection.
